(19) United States
(12) Patent Application Publication
(10) Pub. No.: US 2019/0096532 A1
(43) Pub. Date: Mar. 28, 2019

(54) METHOD AND SYSTEM FOR TRACKING ILLNESS

(71) Applicant: Sickweather, LLC, Windsor Mill, MD (US)

(72) Inventor: Graham Dodge, Windsor Mill, MD (US)

(73) Assignee: Sickweather, LLC, Windsor Mill, MD (US)

(21) Appl. No.: 16/143,005

(22) Filed: Sep. 26, 2018

Related U.S. Application Data
(60) Provisional application No. 62/563,396, filed on Sep. 26, 2017.

(51) Int. Cl. G16H 50/80; H04W 4/029

(52) U.S. Cl. CPC G16H 50/80; H04W 4/029

(57) ABSTRACT

A system for tracking a spread of an illness is described herein. The system includes one or more processors configured to receive disease information of a user, a pseudonymous identifier associated with the user, and a position information of the user from a mobile device of the user, the mobile device being configured to communicate with the one or more processors. The one or more processors are configured to track a location of the user based on the position information and the pseudonymous identifier. The pseudonymous identifier contains anonymous data about the user.

### PRIORITY

This application claims priority to U.S. Provisional Patent Application No. 62/563,396, filed Sep. 26, 2017, the contents of which are incorporated herein in their entirety.

### CROSS-REFERENCE TO RELATED APPLICATIONS

The entire contents of applicant's co-pending U.S. application Ser. No. 14/123,923, filed on Dec. 4, 2013, are expressly incorporated herein by reference.

### FIELD

The present invention relates to tracking illnesses in general, and in particular to a method and system to track an illness using a pseudonymous identifier associated with a user.

### INTRODUCTION

Various methods, systems and techniques currently exist for tracking the spread of illness or disease. One of such techniques includes for example a system with mobile application named “Influ” for tracking the spread of influenza (flu). The Influ application is based on Rich Site Summary (RSS) feeds from the United States Centers for Disease Control and Prevention (CDC). Influ is a mobile phone app and web service for reporting and tracking the spread of flu, all in real time and with spatial resolution. Reports are submitted through a simplified, text-free, graphical interface. Reports are displayed on an interactive map as high resolution geolocation data, so users can make real-time practical decisions to maintain their health based on disease activity near them. Influ also uses a scoring system to let users track how they are doing at limiting the spread of disease and maintaining their health, whether it is staying at home if they are sick, staying away from nearby disease hotspots, or using the service regularly. The application combines real-time user reports with a built-in RSS feed reader for keeping track of developments about the flu and other diseases using the CDC's RSS feeds and other news sites on the web.

Other methods and systems also exist that are geared towards controlling the spread of a communicable disease. Physiological data and location data received from a user via a mobile device is used to estimate the likelihood that an individual is ill. The user is tracked using cookies and/or personal information of the user and location information of the user such as GPS coordinates, address information, and other known location information. Other methods and systems include collecting, tracking, and dissemination of health information where a health care provider can input disease information into a disease tracking platform.

The above methods and systems have a limited usage and have various problems including lacking the support to maintain the anonymity of the user while still providing the ability to track the spread of a disease. Therefore, a need remains for a system that solves the above and other problems associated with disease tracking.

### SUMMARY

An aspect of the present disclosure is to provide a system for tracking a spread of an illness. The system includes one or more processors configured to receive disease information of a user, a pseudonymous identifier associated with the user, and a position information of the user from a mobile device of the user, the mobile device being configured to communicate with the one or more processors. The one or more processors are configured to track a location of the user based on the position information and the pseudonymous identifier. The pseudonymous identifier contains anonymous data about the user.

Another aspect of the present disclosure is to provide a mobile device having an application running thereon to determine a spread of a disease. The mobile device includes an input device configured to receive an input from a user including disease information of the user, a pseudonymous identifier generator configured to generate a pseudonymous identifier associated with the user, a position unit configured to provide position information of the user, and a processor unit configured to communicate the disease information of the user, the pseudonymous identifier associated with the user, and the position information of the user to a server computer. The server computer is configured to track a location of the user based on the position information and the pseudonymous identifier. The pseudonymous identifier contains anonymous data about the user.

### BRIEF DESCRIPTION OF THE DRAWINGS

The present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

FIG. 1 is schematic diagram of a system for tracking illness, according to an embodiment of the present disclosure;

FIG. 2 depicts a screenshot of an embodiment of an application running on a user's mobile device, according to an embodiment of the present disclosure;

FIG. 3 is a screenshot showing examples of data returned and searchable by a server computer, according to an embodiment of the present disclosure; and

FIG. 4 is a screenshot showing examples of other types of data returned and searchable by the server computer, according to an embodiment of the present disclosure.

### DETAILED DESCRIPTION

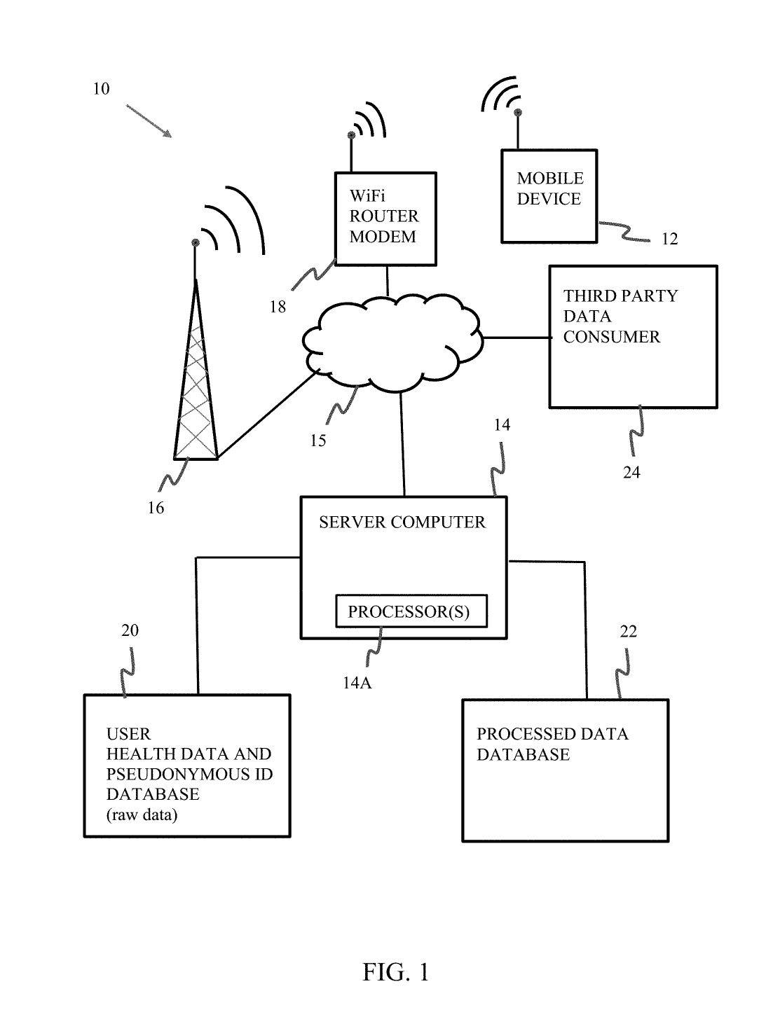

FIG. 1 is schematic diagram of a system 10 for tracking illness, according to an embodiment of the present disclosure. As shown in FIG. 1, the system 10 includes a software application that runs on a mobile device 12 (e.g., a smartphone, a tablet, a laptop, or other mobile device). The application running on the mobile device 12 is configured to enable one or more users to input his health status without any personal identifiable information. A user can report or input anonymously an illness (e.g., a flu or food borne disease) into the mobile device 12, and the application running on the mobile device 12 associates the user input illness with a pseudonymous ID associated with the user. The pseudonymous ID can include, for example, a Mobile Advertising Identifier (MAID), or a Mobile Equipment Identifier (MEID), or both. A Pseudonymous ID is an identifier that does not directly reveal personally identifiable information (PH) of a user.

MAIDs are identifiers that mobile application developers can use to identify who is using their mobile applications. There are three types of MAIDs presently supported by mobile advertisers applications: Apple's Advertising Identifier (IDFA) which is an advertising ID that Apple provides as part of iOS; Android's Advertising ID (AAID) which is an advertising ID that Google provides as part of Android; and Facebook Application User IDs (FAUID) which is an ID corresponding to someone who uses an app that can be retrieved through the Facebook Software Developer's Kit (Facebook SDK).

A mobile equipment identifier (MEID) is a globally unique number identifying a mobile device. The number format is defined by the 3GPP2 report S.R0048 but in practical terms, it can be seen as an International Mobile Equipment Identity (IMEI) but with hexadecimal digits. A MEID is 56 bits long (14 hex digits). It has three fields, including an 8-bit regional code (RR), a 24-bit manufacturer code, and a 24-bit manufacturer-assigned serial number. The check digit (CD) is not considered part of the MEID.

In an embodiment, the MAID and/or MEID information can be provided from third party providers including companies such as Live Ramp, Acxiom and others.

The pseudonymous ID may further include user name handle, and/or other anonymous or anonymized identifiers. The term anonymized is used herein to mean rendered anonymous. For example, according to an embodiment, the user's real name can be anonymized or rendered anonymous by the application running on the mobile device 12 automatically creating an anonymous name handle using the spelling letters of the user's real name along with randomly generated numbers to create a unique user ID, hash or token that will be associated with that user without revealing personally identifiable information (PII) of the user.

The system 10 further includes a server computer 14 including one or more processors 14A. The one or more processors 14A are configured to process information including the health status of the user and the pseudonymous ID received from the mobile device 12. The mobile device 12 is configured to communicate with the server computer 14 through network 15. The network 15 can be any type of network including the internet. For example, as shown in FIG. 1, the mobile device 12 can communicate data with a cellular communication tower and base station 16 which in turn communicates the data through the internet 15 to the server computer 14. Alternatively, in another embodiment, the mobile device 12 can communicate data wirelessly to a WiFi router/modem 18 which in turn communicates the data through the internet 16 to the computer server 14.

The application running on the mobile device 12 associates the illness data input by the user with the MEID and or MAID information to form a unique data packet associated with the user, and the mobile device 12 sends this unique data packet to the server computer 14. According to an embodiment, the application running on the mobile device 12 also tracks the user's location using a location tracking feature such as using the global positioning system (GPS) on the device 12 or using cellular tower triangulation. The mobile device 12 also sends the position information along with the unique data packet to the server computer 14. According to an embodiment, the server computer 14 receives a data packet including the health information of the user, the pseudonymous ID including the MAID data or MAID data, or both, associated with the user, along with position information of the user from the mobile device 12. Based on the data packet containing the health information, the pseudonymous ID including the MAID/MEID information, and the position information received from the user's mobile device 12, the server computer 14 processes the data to track the movement of the user having the illness.

According to an embodiment, the server computer 14 receives a plurality of data packets along with the associated position information from a plurality of users. As a result, the server computer can construct a map of the spread of a specific illness geographically. This can be performed using known statistical and/or graphical techniques. In an embodiment, for example, the data representing a user having an illness can be represented on a graphical user interface (GUI) by a dot superimposed on a geographical map. This data can be, for example, displayed by the server computer 14 on a display device associated with the server computer 14. The server computer 14 can also construct a spread of a specific illness demographically if demographic data is available and gathered by the application running on the mobile device 12 and received by the server computer 14. For example, in an embodiment, the application can obtain the user's demographic information from third party entities based on the user's pseudonymous ID, and use the demographic information to further predict the spread of the illness. The demographic information provided by these third party entities can be cross referenced with certain diseases and conditions that are more strongly associated with certain demographics such as ethnicity, age, gender and household income to build a more accurate infectious disease model. Furthermore, the server computer 14 can also construct a spread of the specific illness in time. According to this embodiment, the mobile device 12 user will continue reporting an illness to the server computer 14 by sending a unique packet containing the illness and the MAID and/or MEID along with an updated position of the user periodically to the server computer 14. The server computer 14 can thus be able to construct the movement of the user having the disease in time. This same operation can be performed for a plurality of users. As a result, the server computer 14 is able to construct from the data gathered from the plurality of users a spread of a selected disease in time.

In an embodiment, the server computer may also be configured to track the spread of illness backwards to identify a geographical origin of the illness or patient zero by isolating previous locations, illnesses, and interests associated with the pseudonymous IDs within the timeframe of the associated viral life cycle. In an embodiment, the server computer 14 may further utilize user's demographic information (e.g., associated with their pseudonymous ID) to more accurately predict the spread of illness.

The system 10 tracks a plurality of users, each user having a personal mobile device 12 and each user having input a disease into the application running on each user's mobile device 12. The disease input by the users can be the same or different diseases. In embodiment, the server computer 14 sends alerts to the plurality of users. For example, the alerts can indicate illness spreading around users' locations. For example, as a user moves from one location to another, the user will be able to receive alerts on the presence of people with an illness (e.g., flu) in the user's present location. Additionally, a user either associated or unassociated with the system 10 may receive an alert that he or she has been exposed to someone infected with an illness.

In an embodiment, the health data input by users along with the pseudonymous (including MAID/MEID) information is collected by the server computer 14 and stored in a pseudonymous identifier database 20 in communication with the server computer 14. The server computer 14 can generate a sick score to provide weighted trends and third party validation. A sick score is a numerical risk value index of sickness. In an embodiment, a sick score may be a numerical value between 0-100 calculated based on number of illness reports in a geographic area multiplied by their associated reproductive scores and divided into the population and/or normalized into a 0-100 range. A reproductive score is a relative value associated with an illness to measure how contagious that illness may be. The health data and associated pseudonymous identifier data (raw data) stored in database 20 can be used by the server computer 14 to determine and monitor the spread of a given disease or illness geographically and in time. The health data and associated pseudonymous identifier data can be used by the server computer 14 to forecast the spread of the disease in time and in location and output processed data of a potential spread of the illness. In an embodiment, this processed data can be stored in processed data database 22 for later consumption.

In an embodiment, the server computer 14 can be configured to identify an individual path and intersections of symptomatic users throughout their journey. This can be accomplished using a searchable interface that can show where the various users have been, who they have interacted with, and where they are going (e.g., using a forecast function). According to an embodiment, the users' movements and disease spread map can be developed without relying on any personal identifiable information from the individual users. Therefore, embodiments of the present system 10 ensure that privacy of the individual users is maintained.

In an embodiment, the server computer 14 can be configured to implement a real-time elastic search tracking system to find “patient zero.” “Patient zero” is the initial patient that contracted the illness or infected by a viral or a bacterial outbreak. Patient zero is the carrier of a communicable disease in an outbreak of the disease. For example, the server computer 14 can also be configured to back-track in time, similar to a rewind operation in a movie, to determine a first instance of apparition of a disease indicating the origin of the disease.

In an embodiment, the computer server 14 can be configured to search the plurality of users who have reported a selected disease symptoms (e.g., flu symptoms), then filter the users having the selected disease by time and/or location, to see where and when the users first became symptomatic. According to an embodiment, this can be done anywhere around the world.

In an embodiment, the computer server 14 can provide the above functionalities to the application running on the user's mobile device 12. In this case, a user in the plurality of users, not necessarily having illness symptoms, may be able to use the application running on his/her mobile device 12 in communication with the server computer 14 to determine where users having a selected disease or symptoms (e.g., flu) are located. Furthermore, the user can also use the application running on the mobile device 12 in communication with the server computer 14 to receive forecast data from the server computer 14 indicating where a potential disease (e.g., flu) may spread in the future. The user then can take appropriate measures to protect him or herself from acquiring the diseases (e.g., flu) or completely avoid the geographical area where the disease is forecasted to be spreading. For example, the user may avoid a certain geographical area where a disease is spreading or the user may elect to take a vaccine (e.g., a flu shot) or wash their hands or see a doctor for diagnosis at the earliest sign of illness to be prescribed an anti-viral or anti-biotic medication.

In an embodiment, the server computer 14 can also be configured to send alerts to all users using a mobile device (e.g., a smartphone) when the users have come in contact with symptomatic users, in a similar fashion as smartphones receiving alert for emergencies, Amber alerts, and severe weather alerts.

In an embodiment, the health data and associated pseudonymous identifier data including MAID and MEID data (raw data) or the processed data can be distributed in the market place or provided to third party consumers 24, such as public health organizations and federal Agencies, or output to pharma companies, insurance companies, retailers, and other health care systems.

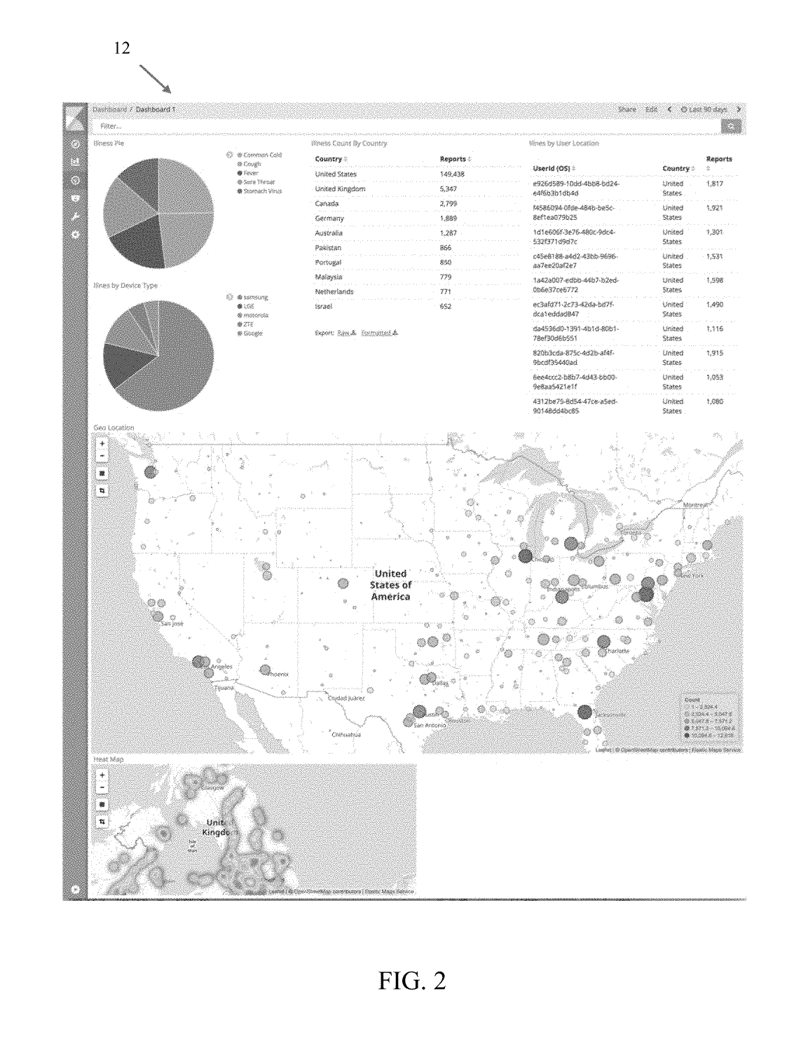

FIG. 2 depicts a screenshot of an embodiment of the application running on the user's mobile device 12. FIG. 2 shows the dashboard graphical user interface (GUI) using a small sample of data. The example GUI shows pie charts of illnesses reported into the system, the device types from which they were reported, the pseudonymous IDs associated with those users whose locations were tracked, a map of where those users were tracked, and various tools for filtering, searching and customizing views of the data.

In an embodiment, the MAID and/or MEID information or data can be provided from third party providers including companies such as Live Ramp, Acxiom and others. In another embodiment, the MAID and/or MEID data can be gathered from other sources or captured by the server computer 14. Open source Elasticsearch framework by Elastic Company can be implemented to run on server computer 14. Elasticsearch is a distributed, multitenant-capable full-text search engine that allows all the data captured by the server computer 14 to be quickly searchable.

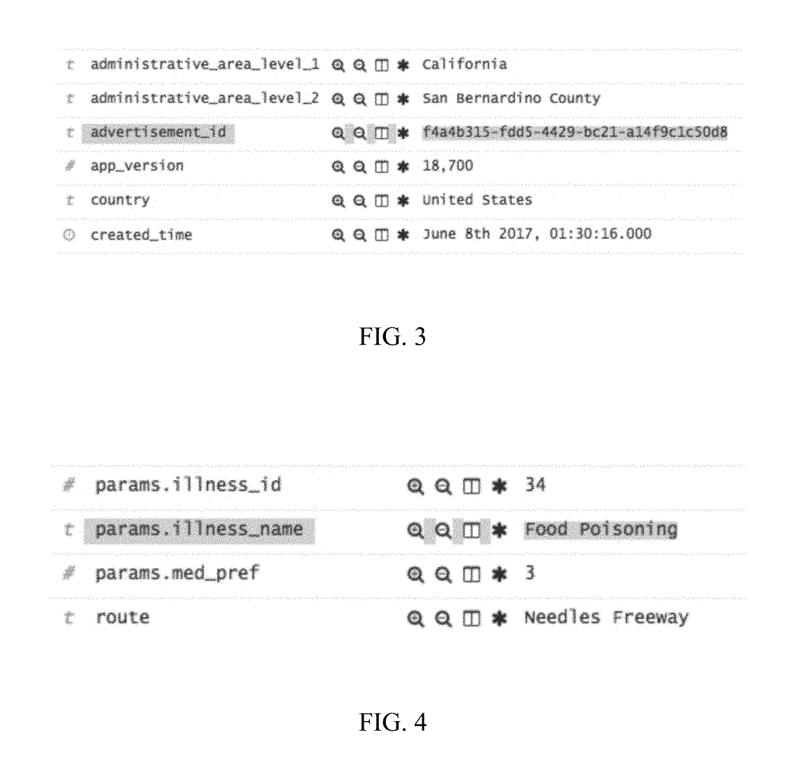

FIG. 3 is a screenshot showing examples of data returned and searchable by the server computer 14, according to an embodiment of the present disclosure. For example, FIG. 3 shows the MAID field (highlighted) of a particular user. The identifier includes a series of letters and numbers to uniquely identify the user. According to an embodiment, the identifier is also stored by third party providers of MAIDs who may have other data on the user, such as ethnicity, age, gender, interests, shopping habits, and more. In this example, fields are populated with the general location of the user logged either at the time of the reporting of the illness or when the user's location was sent by the mobile device 12 and received by the server computer 14 (administriavtive_area_level 1, administrative_area_level 2, country and created time) as well as the advertisement_ID being isolated/filtered from other users and data and that user's app_version of the application running on the user's mobile device 12.

FIG. 4 is a screenshot showing examples of other types of data returned and searchable by the server computer 14, according to an embodiment of the present disclosure. For example, FIG. 4 shows the illness reported by a user on the application running on the mobile device 12. In this example, the user is shown reporting “Food Poisoning” as the disease or illness. FIG. 4 also shows the route (e.g., “Needles Freeway”) taken by the user when the user reported the illness “Food Poisoning.” In an embodiment, the user's location can be used to identify where the user was (in time and/or space) when the user likely contracted the illness. In this example, fields are populated with the illness information and medical preference submitted by the user to the application running on the mobile device 12 (params.illness_id, params.illness_name, params.med_pref) and the specific route or street the user was on at the time of the report. Furthermore, in this example, illness_id 34 indicates “Food Poisoning” as noted in illness_name, and med_pref 3 indicates that the user identifies as preferring both western and alternative forms of medical intervention.

The present system 10 can provide an accurate, predictive, and meaningful sickness forecast geographically (e.g., in the globe) and in time which ultimately results in reducing healthcare costs and saving lives. Embodiments of the system employ, among other things, the tracking capabilities in mobile devices (e.g., smartphones) to trace back a disease outbreak to its original source. Furthermore, embodiments of the present system 10 can similarly track outbreaks and enable quick and precise response and remediation while offering preventative measures with real-time feedback to the general public.

The term “computer system” or “computer server” is used herein to encompass any data processing system or processing unit or units. The computer system or computer server may include one or more processors or processing units. The computer system can also be a distributed computing system. The computer system may include, for example, a desktop computer, a laptop computer, a mobile computing device such as a PDA, a tablet, a smartphone, etc. A computer program product or products may be run on the computer system to accomplish the functions or operations described in the above paragraphs. The computer program product includes a computer readable medium or storage medium or media having instructions stored thereon used to program the computer system to perform the functions or operations described above. Examples of suitable storage medium or media include any type of disk including floppy disks, optical disks, DVDs, CD ROMs, magnetic optical disks, RAMs, EPROMs, EEPROMs, magnetic or optical cards, hard disk, flash card (e.g., a USB flash card), PCMCIA memory card, smart card, or other media. Alternatively, a portion or the whole computer program product can be downloaded from a remote computer or server via a network such as the internet, an ATM network, a wide area network (WAN) or a local area network.

Stored on one or more of the computer readable media, the program may include software for controlling both the hardware of a general purpose or specialized computer system or processor. The software also enables the computer system or processor to interact with a user via output devices such as a graphical user interface, head mounted display (HMD), etc. The software may also include, but is not limited to, device drivers, operating systems and user applications. Alternatively, instead or in addition to implementing the methods described above as computer program product(s) (e.g., as software products) embodied in a computer, the method described above can be implemented as hardware in which, for example, an application specific integrated circuit (ASIC) or graphics processing unit or units (GPU) can be designed to implement the method or methods, functions or operations of the present disclosure.

The various databases described herein may be, include, or interface to, for example, an Oracle™ relational database sold commercially by Oracle Corporation. Other databases, such as Informix™, DB2 (Database 2) or other data storage, including file-based, or query formats, platforms, or resources such as OLAP (On Line Analytical Processing), SQL (Standard Query Language), a SAN (storage area network), Microsoft Access™ or others may also be used, incorporated, or accessed. The database may comprise one or more such databases that reside in one or more physical devices and in one or more physical locations. The database may store a plurality of types of data and/or files and associated data or file descriptions, administrative information, or any other data.

1. A system for tracking a spread of an illness comprising:
one or more processors configured to receive disease information of a user, a pseudonymous identifier associated with the user, and a position information of the user from a mobile device of the user, the mobile device being configured to communicate with the one or more processors, the one or more processors being configured to track a location of the user based on the position information and the pseudonymous identifier,
wherein the pseudonymous identifier contains anonymous data about the user.

2. The system according to claim 1, wherein the pseudonymous identifier includes a Mobile Advertising Identifier (MAID) data, and/or Mobile Equipment Identifier (MEID) data.

3. The system according to claim 2, wherein the MAID includes at least one of an Apple Advertising Identifier (IDFA), an Android Advertising ID (AAID), or a Facebook Application User IDs (FAUID).

4. The system according to claim 2, wherein the MEID is a globally unique number identifying the mobile device of the user.

5. The system according to claim 1, wherein the pseudonymous identifier is received from a third party provider.

6. The system according to claim 1, wherein the position information of the user is obtained from a global positioning system (GPS) unit of the mobile device.

7. The system according to claim 1, wherein the one or more processors are further configured to receive demographic data of the user and process the demographic data to predict a spread of the disease.

8. The system according to claim 1, wherein the one or more processors are configured to construct a spread of the disease in time based on a movement of a plurality of users reporting the disease.

9. The system according to claim 8, wherein the one or more processors are configured to track the spread of the disease backward in time to identify a geographical origin of an occurrence of the disease and/or to identify patient zero.

10. The system according to claim 9, wherein the one or more processors are configured to identify a geographical origin of the occurrence of the disease and/or patient zero by isolating previous locations, illnesses, and interests associated with pseudonymous identifiers within a timeframe of an associated viral cycle.

11. The system according to claim 1, further comprising a health data and pseudonymous identifier database in communication with the one or more processors, the health data and pseudonymous identifier database being configured to store the pseudonymous identifier.

12. The system according to claim 1, further comprising a processed data database in communication with the one or more processors, the processed data database being configured to store processed data comprising data forecast of a spread of a disease determined based on the position information and the pseudonymous identifier of the user.

13. A mobile device having an application running thereon to determine a spread of a disease, the mobile device comprising:
an input device configured to receive an input from a user including disease information of the user;
a pseudonymous identifier generator configured to generate a pseudonymous identifier associated with the user;
a position unit configured to provide position information of the user; and
a processor unit configured to communicate the disease information of the user, the pseudonymous identifier associated with the user, and the position information of the user to a server computer, the server computer being configured to track a location of the user based on the position information and the pseudonymous identifier,
wherein the pseudonymous identifier contains anonymous data about the user.

14. The mobile device of claim 13, wherein the pseudonymous identifier includes Mobile Advertising Identifier (MAID) data and/or Mobile Equipment Identifier (MEID) data.

15. The mobile device according to claim 13, wherein the processor unit is configured to receive processed data from the server computer, the processed data including a spread of a disease determined based on the position information and the pseudonymous identifier of the user.

16. The mobile device according to claim 15, wherein the processed data includes a spread of the disease in time based on a movement of a plurality of users reporting the disease to the server computer.

17. The mobile device according to claim 14, wherein the MAID includes at least one of an Apple Advertising Identifier (IDFA), an Android Advertising ID (AAID), or a Facebook Application User IDs (FAUID).

18. The mobile device according to claim 14, wherein the MEID is a globally unique number identifying the mobile device of the user.

19. The mobile device according to claim 14, wherein at least one of the MAID or the MEID is received from a third party provider.

20. The mobile device according to claim 13, wherein the position unit comprises a global positioning system (GPS) unit.
